(19) United States
(12) Patent Application Publication
(10) Pub. No.: US 2025/0037793 A1
(43) Pub. Date: Jan. 30, 2025

(54) PHYLOGENETIC PLACEMENT USING TAXONOMY-INDEPENDENT FEATURE GENERATION

(71) Applicant: REGENTS OF THE UNIVERSITY OF MICHIGAN, Ann Arbor, MI (US)

(72) Inventors: Jonathan Golob, Ann Arbor, MI (US); Marina Sirota, Belmont, CA (US)

(21) Appl. No.: 18/786,332

(22) Filed: Jul. 26, 2024

Related U.S. Application Data
(60) Provisional application No. 63/529,121, filed on Jul. 26, 2023.

(51) Int. Cl. G16B 20/20; G16B 40/30; G16B 50/30; G16H 50/30

(52) U.S. Cl. CPC G16B 20/20; G16B 40/30; G16B 50/30; G16H 50/30

(57) ABSTRACT

Methods and systems for generating improved taxonomy-independent, generalizable features of alleles. The systems and methods may include (1) receiving a plurality of amplicon sequence variants corresponding to one or more microorganism communities; (2) generating a de novo phylogenetic tree representing a plurality of full-length and non-clustered alleles and the plurality of amplicon sequence variants; (3) generating a set of one or more phylogenetically-binned amplicon sequence variants (phylotypes) by a divide-and-conquer strategy; and/or (4) storing the set of phylotypes in one or more computer memories.

### CROSS-REFERENCE TO RELATED APPLICATIONS

The present application claims priority to U.S. Provisional Application No. 63/529,121, entitled PHYLOGENETIC PLACEMENT USING TAXONOMY-INDEPENDENT FEATURE GENERATION, filed on Jul. 26, 2023, and hereby incorporated by reference in its entirety.

### BACKGROUND

The human body contains a diverse ecosystem of microorganisms that play a crucial role in human health. However, making meaningful associations between particular microorganisms and particular human health aspects has continued to be a challenge. This is due, in large part, to a lack of robust, generalizable and biologically meaningful features in the identification and classification of microorganisms.

Two conventional approaches for harmonizing microbiological data across independent studies into a feature set are closed operational taxonomic units (cOTUs) and taxonomy. However, both approaches suffer from being heavily dependent upon reference sets and are limited in precision relative to underlying biology.

Another conventional approach for harmonizing microbiological data is phylogenetic placement, but it suffers from a lack of validated, count-based features that are suitable for use in computational processing. Generally, the existing conventional approaches are not fine-grained enough be useful for developing predictive clinical models or post hoc harmonization.

Therefore, improved methods for phylogenetic placement using taxonomy-independent feature generation are needed.

### SUMMARY

In one aspect, a computer-implemented method for a generation of improved taxonomy-independent, generalizable features may be provided. The method may include: (1) receiving, via one or more processors, a plurality of amplicon sequence variants corresponding to one or more microorganism communities; (2) generating, via one or more processors, a de novo phylogenetic tree representing a plurality of full-length and non-clustered alleles and the plurality of amplicon sequence variants; (3) generating, via one or more processors, a set of one or more phylogenetically-binned amplicon sequence variants (phylotypes) by a divide-and-conquer strategy that may include the steps of: (a) assigning each of the plurality of amplicon sequence variants to one or more pre-groups according to a respective location within the de novo phylogenetic tree of one or more of the plurality of amplicon sequence variants; (b) determining, for each of the pre-groups of the plurality of amplicon sequence variants, a respective lowest common ancestor; (c) determining pre-group pairwise distances by computing, for each lowest common ancestor, a respective lowest common ancestor phylogenetic distance to each lowest common ancestor of each pre-group; (d) generating, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance; (e) determining group pairwise distances by computing, for each of the plurality of amplicon sequence variants within each of the groups, a respective amplicon sequence variant phylogenetic distance to each of the amplicon sequence variants of each group; and/or (f) generating, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and/or (4) storing, via one or more processors, the set of phylotypes in one or more computer memories. The method may include additional, less, or alternate actions, including those discussed elsewhere herein.

In another aspect, a computer system for a generation of improved taxonomy-independent, generalizable features may be provided. The computing system may include one or more processors and associated transceivers, and a non-transitory program memory coupled to the one or more processors and storing executable instructions that, when executed by the one or more processors, cause the computer system to: (1) receive a plurality of amplicon sequence variants corresponding to one or more microorganism communities; (2) generate a de novo phylogenetic tree representing a plurality of full-length and non-clustered alleles and the plurality of amplicon sequence variants; (3) generate a set of one or more phylogenetically-binned amplicon sequence variants (phylotypes) by a divide-and-conquer strategy that cause the one or more processors to: (a) assign each of the plurality of amplicon sequence variants to one or more pre-groups according to a respective location within the de novo phylogenetic tree of one or more of the plurality of amplicon sequence variants; (b) determine, for each of the pre-groups of the plurality of amplicon sequence variants, a respective lowest common ancestor; (c) determine pre-group pairwise distances by computing, for each lowest common ancestor, a respective lowest common ancestor phylogenetic distance to each lowest common ancestor of each pre-group; (d) generate, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance; (e) determine group pairwise distances by computing, for each of the plurality of amplicon sequence variants within each of the groups, a respective amplicon sequence variant phylogenetic distance to each of the amplicon sequence variants of each group; and/or (f) generate, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and/or (4) store the set of phylotypes in one or more computer memories. The computer system may be configured to include additional, less, or alternate functionality, including that discussed elsewhere herein.

In yet another aspect, a tangible, a non-transitory computer-readable medium for a generation of improved taxonomy-independent, generalizable features may be provided. The executable instructions, when executed by one or more processors of a computer system, may cause the computer system to: (1) receive a plurality of amplicon sequence variants corresponding to one or more microorganism communities; (2) generate a de novo phylogenetic tree representing a plurality of full-length and non-clustered alleles and the plurality of amplicon sequence variants; (3) generate a set of one or more phylogenetically-binned amplicon sequence variants (phylotypes) by a divide-and-conquer strategy that cause the one or more processors to: (a) assign each of the plurality of amplicon sequence variants to one or more pre-groups according to a respective location within the de novo phylogenetic tree of one or more of the plurality of amplicon sequence variants; (b) determine, for each of the pre-groups of the plurality of amplicon sequence variants, a respective lowest common ancestor; (c) determine pre-group pairwise distances by computing, for each lowest common ancestor, a respective lowest common ancestor phylogenetic distance to each lowest common ancestor of each pre-group; (d) generate, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance; (e) determine group pairwise distances by computing, for each of the plurality of amplicon sequence variants within each of the groups, a respective amplicon sequence variant phylogenetic distance to each of the amplicon sequence variants of each group; and/or (f) generate, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and/or (4) store the set of phylotypes in one or more computer memories. The instructions may direct additional, less, or alternate functionality, including that discussed elsewhere herein.

### BRIEF DESCRIPTION OF THE DRAWINGS

The figures described below depict various embodiments of the systems and methods disclosed herein. It should be understood that the figures depict illustrative embodiments of the disclosed systems and methods, and that the figures are intended to be exemplary in nature. Further, wherever possible, the following description refers to the reference numerals included in the following figures, in which features depicted in multiple figures are designated with consistent reference numerals.

There are shown in the drawings arrangements which are presently discussed, it being understood, however, that the present embodiments are not limited to the precise arrangements and instrumentalities shown, wherein:

FIG. 1A depicts example primers for the nine hypervariable regions of the 16S rRNA gene;

FIG. 1B depicts an example subclade of a phylogenetic tree of full-length 16S rRNA alleles;

FIG. 2A depicts an example pre-grouping of the exemplary subclade of the phylogenetic tree of full-length 16S rRNA alleles;

FIG. 2B depicts an example matrix of pair-wise distances between lowest common ancestors of the exemplary subclade of the phylogenetic tree of full-length 16S rRNA alleles;

FIG. 3A depicts an example grouping of the example pre-grouping of the exemplary subclade of the phylogenetic tree of full-length 16S rRNA alleles;

FIG. 3B depicts an example matrix of pair-wise distances between amplicon sequence variants within groups of the exemplary subclade of the phylogenetic tree of full-length 16S rRNA alleles;

FIG. 4 depicts example phylotype categorizations of the alleles;

FIG. 5 depicts an example post hoc integration of novel alleles;

FIG. 6 depicts empirical results of ASVs phylotyped at varying threshold phylogenetic distances and alleles taxonomy classified at varying taxonomic levels compared to a full dereplicated allele;

FIG. 7 depicts an example server for the implementation of the methods and systems described herein;

FIG. 8 depicts an example computing environment for the implementation of the methods and systems described herein; and

FIG. 9 depicts an example method for the generation of improved taxonomy-independent, generalizable features, according to some aspects.

The figures depict the present embodiments for purposes of illustration only. One skilled in the art will readily recognize from the following discussion that alternate embodiments of the structures and methods illustrated herein may be employed without departing from the principles of the invention described herein.

### DETAILED DESCRIPTION

Described herein are improvements to the phylogenetic placement of microbiological data. Phylogenetic placement is the process of placing variants of a particular sequence (or an “allele”) of a polynucleotide chain (e.g., ribonucleic acid (RNA) or deoxyribonucleic acid (DNA)) onto a hierarchical tree graph indicating a hypothetical evolution of mutations of the alleles over time. Artificial alleles are known as “amplicons,” and so herein the terms “alleles,” “amplicons,” and “amplicon sequence variants” (ASVs) are used interchangeably. In a phylogenetic tree, the leaf nodes are the alleles—gathered from (i) real-world data, (ii) synthetic replication, or (iii) in silico (e.g., virtually simulated)—and the interior nodes are a combination of alleles and inferred, hypothetical, ancestral alleles.

Once the phylogenetic tree has been generated, closely related alleles on the phylogenetic tree may be compared. This comparison, also known as a “pair-wise distance measure” or a “pair-wise similarity measure,” can then be used to categorize (or “bin”) the alleles. This resulting categorization is defined herein as a “phylotype.”

In some embodiments, the binning of alleles into phylotypes may be performed by a divide-and-conquer strategy to optimize the algorithm. In this embodiment, lowest common ancestors (LCAs) of the phylogenetic tree may be determined. Then, pair-wise phylogenetic distancing—in conjunction with a threshold phylogenetic distance value—may be performed on the LCAs to group the alleles of similar LCAs. Once grouped, pair-wise phylogenetic distancing—in conjunction with another threshold phylogenetic distance value (or in some embodiments, the same threshold phylogenetic distance value)—may be performed again, this time on the individual alleles within each group.

In this way, the generation of phylotypes is estimated to be in approximately O(n log n) time (as opposed to a naive comparison of all alleles, which would be O(n²) time). This optimization is due to the hierarchical structure of phylogenetic trees. Because alleles of similar sequences can be grouped together via the phylogenetic tree, the pair-wise distance comparisons can be broken down into two stages that can run in parallel via recursion and/or parallel processing. In general, the divide-and-conquer approaches of the present techniques divide the problem of pairwise distance comparisons into a problem size that reduces by a factor at each iteration or recursive step (e.g., by halving the problem into two subproblems of equal size). This property represents an advantageous improvement over conventional techniques that have less efficient asymptotic runtimes.

Additionally, the recursive and parallel nature of the divide-and-conquer strategy allows for an optimization in terms of memory. Because the strategy employed only compares the pair-wise distances of similar groups of alleles, only the resulting phylotypes need to be stored for further calculations. As such, the memory allocated to perform the pair-wise comparisons within a group may be reallocated to the next group of alleles and so on. Thus, the amount of memory required is greatly reduced from approximately a quadratic (n²) number of memory cells to approximately a linear (n) number of memory cells. The improved asymptotic properties of the present techniques represent multiple advantageous improvements over conventional techniques, by significantly reducing computational overhead for phylotype-based classification, not only in terms of computational cycles (e.g., GPU cycles) but also in terms of storage requirements during training.

Further, newly collected alleles can be integrated easily. By leveraging existing phylogenetic trees, new alleles can be quickly and accurately placed onto branches of best fit and binned. As such, the resulting phylotypes are a predictable and reproducible way of categorizing alleles. As experimentally demonstrated, replicated alleles from various PCR primers were consistently placed on the same sub-pendant as the source allele when placed back onto the phylogenetic tree. In this way, alleles from across studies and clinical tests, regardless of how the alleles were generated or primed, may be harmonized into a single data set.

Phylotypes are a vast improvement over cOTUs and taxonomy as they are predictive of clinical outcomes. In one experiment, a set of phylotypes of human gut microbiota were generated and used to develop a machine learning model for the prediction of an individual's body-mass index (BMI). Once trained and validated, the machine learning model was able to accurately predict the BMI of previously unseen individuals solely on the phylotype categorization of their gut microbiota. Similarly, in another experiment, a set of phylotypes of human vaginal microbiota were generated and used to develop a machine learning model for the prediction of preterm birth and early preterm birth. As with the gut microbiota experiment, the developed machine learning model was able to accurately predict whether a pregnancy would be preterm or be an early preterm solely on the phylotype categorization of the individual's vaginal microbiota.

Thus, phylotypes, and their utilization in machine learning, are a clear improvement in the field as they are a robust, generalizable, and biologically meaningful way to use microbiological data to (i) predict human health aspects and/or (ii) associate microorganisms to human biological functions.

### Exemplary Allele Generation

Phylogenetic placement is particularly effective when applied on polynucleotide chains that are taxonomically informative. For instance, researchers have historically selected the 16S ribosomal RNA (rRNA) strand-derived from prokaryotic ribosomes-when studying the phylogenies of bacteria. However, the methods and systems described herein are not limited to any particular polynucleotide chain or sequences of polynucleotide chains, regardless of whether such chains were harvested, synthesized, or simulated. Similarly, the methods and systems described herein are not limited to any particular microorganism(s). Rather, the present techniques may be applicable to any microorganism (e.g., bacteria, fungi, viruses, etc.). ASVs of 16S rRNA are discussed to illustrate the techniques described herein.

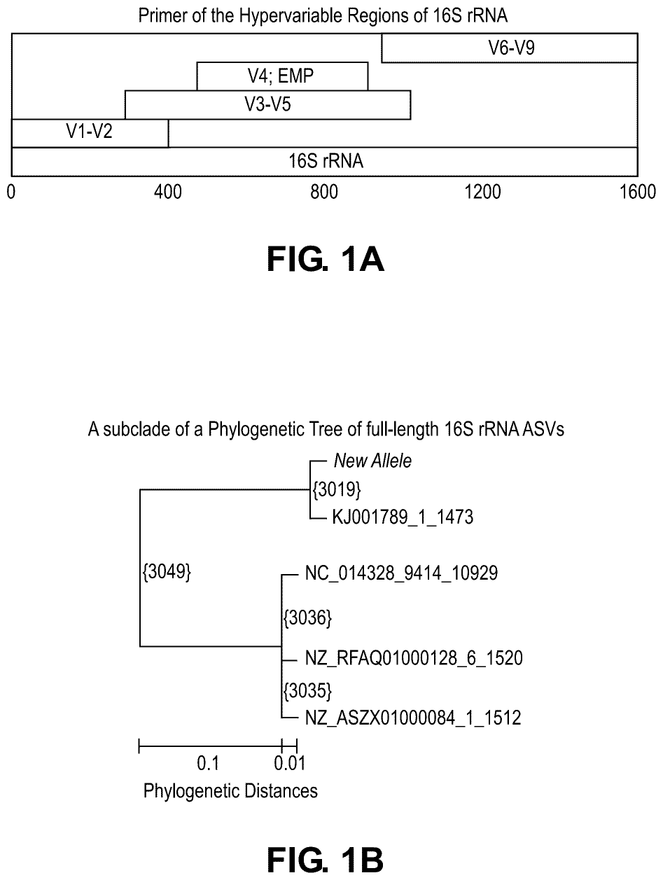

FIG. 1A depicts a strand of 16S rRNA having nine hypervariable regions (V1-V9). FIG. 1A further depicts polymerase chain reaction (PCR) primers of the 16S rRNA. In one illustrative example, a PCR primer targets hypervariable regions V1-V2. As another illustrative example, a PCR primer targets hypervariable regions V3-V5. As yet another illustrative example, a PCR primer targets hypervariable region 4V (Earth Microbiome Project (“EMP”)). And as yet another example, a PCR primer targets hypervariable regions V6-V9.

Using the PCR primers, the corresponding alleles of a bacteria's 16S rRNA can be copied and mass replicated—a process known as high-throughput sequencing.

### Exemplary Phylogenetic Placement

Once a set of alleles corresponding to a particular portion of a strand of RNA have been collected (e.g., ASVs corresponding to 16S rRNA's hypervariable regions of V1-V2), the alleles may be arranged to form a phylogenetic tree.

The methods and systems described herein are not limited to any particular method of constructing a phylogenetic tree. As an illustrative, non-limiting example, the pplacer algorithm of phylogenetic tree construction may be used to demonstrate the techniques described herein.

Pplacer is a two-stage algorithm. The first stage calculates likelihood vectors to determine a set of locations to place an allele onto the phylogenetic tree. The second stage then calculates the posterior probabilities of the locations determined by the first stage. Since the two-stage algorithm performs these calculations for every allele to be added to the phylogenetic tree, the pplacer algorithm runs in approximately linear time (O(n)).

FIG. 1B depicts an example subclade of a phylogenetic tree of 16S rRNA ASVs. The subclade has a phylogenetic distance depth of 0.13 and the alleles have a phylogenetic distance depth of 0.01 from their parent alleles.

It should be noted that a new phylogenetic tree need not be generated every time a new allele is collected. For example, as illustrated in FIG. 1B, a new allele (New Allele) is added to an existing phylogenetic tree and is placed under allele {3019} based on its similarity to allele KJ001789_1_1473.

To demonstrate the accuracy of phylogenetic placement, 10,000 ASVs of a 16S rRNA allele were replicated in silico using four distinct PCR primers (e.g., one targeting hypervariable regions V1-V2, one targeting hypervariable regions V3-V5, V4, and one targeting hypervariable regions V6-V9). The source allele had already been placed onto a phylogenetic tree. When the set of replicated ASVs were placed onto the phylogenetic tree, they were all placed in the correct sub-pendant despite the use of different PCR primers (and notably, the ASVs replicated using the PCR primers targeting the hypervariable regions V6-V9 were placed on the correct leaf node of the phylogenetic tree).

### Exemplary Phylotype Generation

Once the phylogenetic tree has been constructed, the alleles may then be categorized based upon their similarities to every other allele on the tree. It is noted that a pair-wise comparison of every allele may be performed without the generation of a phylogenetic tree. However, such a naïve algorithm would have a quadratic (O(n²)) run time, becoming quickly computationally intractable as the size of n grows. By leveraging the hierarchical structure of a phylogenetic tree, similar alleles may be compared in parallel, reducing the run time of the comparisons such that they become computationally tractable.

The methods and systems described herein are not limited to any particular method of optimization. As an illustrative, non-limiting example, a divide-and-conquer approach may be used to demonstrate the techniques described herein.

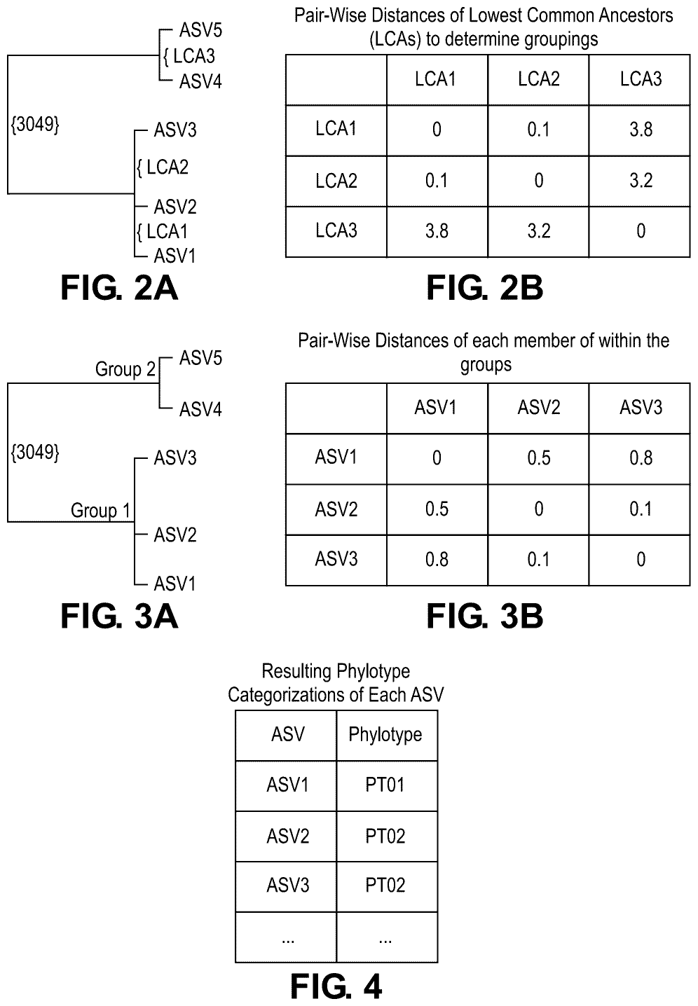

FIG. 2A depicts the example subclade of FIG. 1B relabeled based upon the hierarchical structure of the phylogenetic tree. In particular, the ASVs labelled as ASV1, ASV2, ASV3, ASV4, and ASV5, and the parent alleles that are the lowest common ancestors (LCAs) of the ASVs are identified and labelled as LCA1, LCA2, and LCA.

Using a divide-and-conquer approach, the pair-wise phylogenetic distances between LCAs are generated, as depicted in FIG. 2B. Each pair-wise phylogenetic distance of the LCAs is compared to a threshold phylogenetic distance (e.g., from 0.1 to 1.0) to group the LCAs, as depicted in FIG. 3A. Empirical testing has shown an optimal phylogenetic distance of 0.1. For example, LCA 1 and LCA 2 are grouped together into Group 1 because they have a pair-wise phylogenetic distance of 0.1 which does not exceed the threshold phylogenetic distance of 0.1. Conversely, LCA 3 is placed into a Group 2 because it has a pair-wise phylogenetic distance of 3.8 with LCA 1 and a pair-wise phylogenetic distance of 3.2 with LCA 2, both of which exceed the threshold phylogenetic distance of 0.1.

Once the groups have been generated, the pair-wise distances between each of the ASVs in each group are generated, as depicted in FIG. 3B. Each pair-wise phylogenetic distance of the ASVs of each group is compared to a threshold phylogenetic distance (e.g., 0.1) to group the ASVs by phylotype, as depicted in FIG. 4. ASV2 and ASV3 are both grouped to the same phylotype, PT02, because they have a pair-wise phylogenetic distance of 0.1 which does not exceed the threshold phylogenetic distance of 0.1. Conversely, ASV1 is grouped into a phylotype, PT01, because it has a pair-wise phylogenetic distance of 0.5 with ASV2 and a pair-wise phylogenetic distance of 0.8 with ASV3, both of which exceed the threshold phylogenetic distance of 0.1.

This process of generating phylotypes may be expanded to any sized phylogenetic tree comprising any number of n alleles.

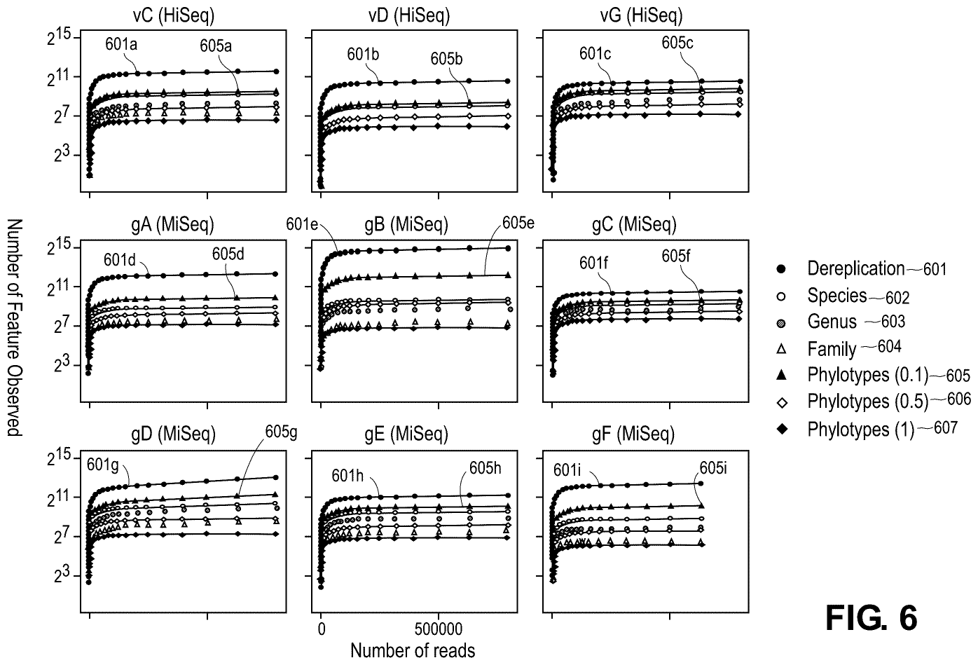

The threshold phylogenetic distances may be empirically determined via experimentation. Using real-world data, amplicons from six gut microorganisms and three vaginal microorganisms may be gathered from research studies and placed onto a phylogenetic tree. The amplicons may then be binned into phylotypes using phylogenetic threshold distances of 1.0, 0.5, and 0.1. The resulting phylotyped alleles and alleles taxonomically classified using traditional methods at differing taxonomic levels (e.g., species, genus, and family) may then be compared to full-length, dereplicated alleles via rarefaction curves. In empirical testing, phylotyped alleles binned using a phylogenetic distance of 0.1 have been demonstrated as the alleles most similar to the dereplicated allele, followed closely by species level taxonomy. As illustrated in FIG. 6, the 0.1 phylotype data lines 605 represent the respective optimum thresholds beyond which no additional classification fidelity is gained through observing additional features as the number of reads increases.

### Exemplary Post HOC Integration of Previously Unseen Alleles

As discussed above, one of the conventional challenges/problems has been the inability to compare microbiome studies due to lack of granularity in microbiome classification methods. The present techniques improve upon conventional classification techniques by leveraging phylogenetic classification into clusters of genetically similar alleles that can then be used to (i) predict human health aspects and/or (ii) associate microorganisms to human biological functions.

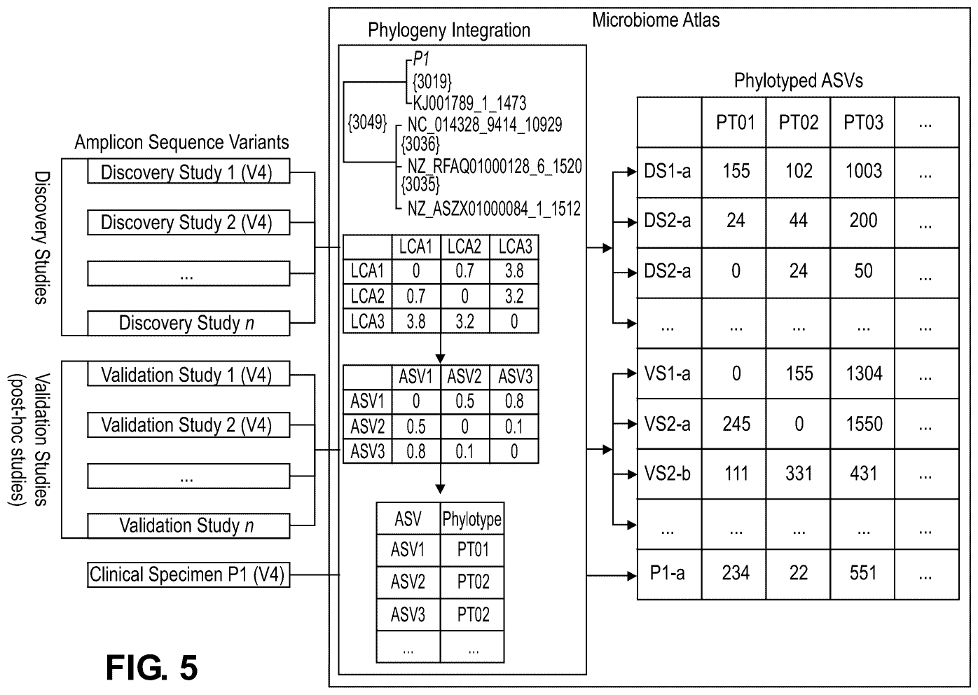

For example, FIG. 5 depicts an example post hoc integration of novel alleles. As illustrated, phylotypes are initially generated on ASVs gathered from discovery and validation studies. Then, novel ASVs (e.g., Clinical Specimen P1) are added to the system and binned by phylotype. As illustrated, P1 is placed onto the pre-existing phylogenetic tree, as illustrated in FIG. 1B. P1 is then pair-wise compared to the other ASVs within its group on the phylogenetic tree. The resulting comparison then either assigns P1 to an existing phylotype—if one of its pair-wise distances does not exceed the threshold phylogenetic distance of 0.1—or to a newly generated phylotype.

Additionally, the phylotypes can be utilized to form a microbiome atlas. In some embodiments, the microbiome atlas is a combination of (i) an existing phylogenetic tree, (ii) a set of existing phylotypes and, (iii) a set of ASVs placed onto the phylogenetic tree and binned into the existing phylotypes. Additionally or alternatively, the microbiome atlas may also include executable instructions on how to generate the existing phylogenetic tree, generate the set of existing phylotypes, place the set of ASVs onto the existing phylogenetic tree, and/or bin the set of ASV into the existing phylotypes. The microbiome atlas may then be transmitted to various laboratories to harmonize new data (such as patient specimens) in a manner consistent with the standards of a clinical molecular biology laboratory.

As depicted in FIG. 5, discovery study ASVs and validation study ASVs are used to generate the phylogenetic tree and phylotypes used in the microbiome atlas. Then, using the Microbiome Atlas, P1 is placed onto the phylogenetic tree under allele {3019} and binned into phylotype PT03. This addition may then update the microbiome atlas in future clinical studies.

### Exemplary Machine Learning Techniques

The present embodiments may involve, inter alia, the use of cognitive computing, predictive modeling, machine learning, and/or other modeling techniques and/or algorithms. In certain embodiments, the systems, methods, and/or techniques discussed herein may use heuristic engines, algorithms, machine learning, cognitive learning, deep learning, combined learning, predictive modeling, and/or pattern recognition techniques. For instance, a processor and/or a processing element may be trained using supervised or unsupervised machine learning, and the machine learning program may employ a neural network, which may be a convolutional neural network (CNN), a fully convolutional neural network (FCN), a deep learning neural network, and/or a combined learning module or program that learns in two or more fields or areas of interest. Machine learning may involve identifying and/or recognizing patterns in existing data in order to facilitate making predictions, estimates, and/or recommendations for subsequent data. Models may be created based upon example inputs in order to make valid and reliable outputs for novel inputs.

Additionally or alternatively, the machine learning programs may be trained and/or validated using labeled training data sets. For example, a data set may include phylotyped microbiological data of individuals with corresponding labels of those individuals' BMI. The machine learning programs may utilize deep learning algorithms that may be primarily focused on pattern recognition and may be trained after processing multiple examples.

In supervised machine learning, a processing element identifies patterns in existing data to make predictions about subsequently received data. Specifically, the processing element is trained using training data, which includes example inputs and associated example outputs. Based upon the training data, the processing element may generate a predictive function which maps outputs to inputs and may utilize the predictive function to generate outputs based upon data inputs. The exemplary inputs and exemplary outputs of the training data may include any of the data inputs or outputs described herein. In some exemplary embodiments, the processing element may be trained by providing it with a large sample of data with known characteristics or features. In this way, when subsequent novel inputs are provided the processing element may, based upon the discovered association, accurately predict the correct output.

In unsupervised machine learning, the processing element finds meaningful relationships in unorganized data. Unlike supervised learning, unsupervised learning does not involve user-initiated training based upon example inputs with associated outputs. Rather, in unsupervised learning, the processing element may organize unlabeled data according to a relationship determined by at least one machine learning method/algorithm employed by the processing element. Unorganized data may include any combination of data inputs and/or outputs as described herein.

In semi-supervised machine learning, the processing element may use thousands of individual supervised machine learning iterations to generate a structure across the multiple inputs and outputs. In this way, the processing element may be able to find meaningful relationships in the data, similar to unsupervised learning, while leveraging known characteristics or features in the data to make predictions.

In reinforcement learning, the processing element may optimize outputs based upon feedback from a reward signal. Specifically, the processing element may receive a user-defined reward signal definition, receive a data input, utilize a decision-making model to generate an output based upon the data input, receive a reward signal based upon the reward signal definition and the output, and alter the decision-making model so as to receive a stronger reward signal for subsequently generated outputs.

In some embodiments, the machine learning model may include a neural network, such as a convolutional neural network (CNN) model and/or a fully convolutional neural network (FCN). For example, the CNN may be trained on a set of labeled historical data to produce a binary classification decision as to whether or not a pregnancy will be an early preterm birth. Accordingly, the training data may include a first set of phylotyped microbiological data that are labeled as pregnancies not resulting in an early preterm birth and a second set of phylotyped microbiological data that are labeled as pregnancies resulting in an early preterm birth.

Generally, the second set of data should include a sufficient number of phylotyped microbiological data of pregnancies resulting in an early preterm birth for the machine learning model to identify characteristics that can be accurately associated with pregnancies resulting in an early preterm birth. For example, there may be multiple distinct ASVs that link to pregnancies resulting in an early preterm birth.

### Exemplary Machine Learning Methods Using Phylotyped Microbiological Data

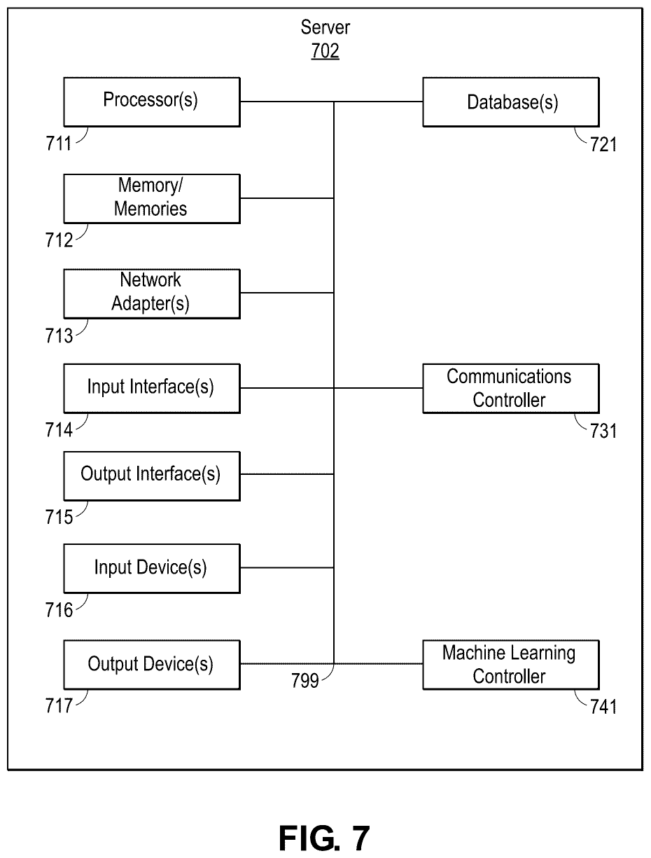

FIG. 7 depicts an exemplary server 702 for the implementation of the methods and systems described herein. In some embodiments, the exemplary server 702 may develop one or more machine learning models to (i) predict human health aspects and/or (ii) associate microorganisms to human biological functions. Additionally or alternatively, in some embodiments the exemplary server 702 may apply one or more pretrained machine learning models on novel data. In some embodiments, the training development of the one or more machine learning models and/or the application of the one or more pretrained machine learning models may be performed by different servers. The exemplary server 702 may include one or more processors 711, one or more memories 712, one or more network adapters 713, one or more input interfaces 714, one or more output interfaces 715, one or more input devices 716, one or more output devices 717, one or more databases 722, one or more communication controllers 731, and/or one or more machine learning controllers 741. Any of the components of the the exemplary server 702 may be communicatively coupled to one another via a communication bus 799.

The one or more processors 711 may be, or may include, one or more central processing units (CPU), one or more coprocessors, one or more microprocessors, one or more graphical processing units (GPU), one or more digital signal processors (DSP), one or more application specific integrated circuits (ASIC), one or more programmable logic devices (PLD), one or more field-programmable gate arrays (FPGA), one or more field-programmable logic devices (FPLD), one or more microcontroller units (MCUs), one or more hardware accelerators, one or more special-purpose computer chips, and one or more system-on-a-chip (SoC) devices, etc.

The one or more memories 712 may be, or may include, any local short term memory (e.g., random access memory (RAM), read only memory (ROM), cache, etc.) and/or any long term memory (e.g., hard disk drives (HDD), solid state drives (SSD), etc.). The memories may store computer-readable instructions configured to implement the methods described herein.

The one or more network adapters 713 may be, or may include, a wired network adapter, connector, interface, etc. (e.g., an Ethernet network connector, an asynchronous transfer mode (ATM) network connector, a digital subscriber line (DSL) modem, a cable modem) and/or a wireless network adapter, connector, interface, etc. (e.g., a Wi-Fi connector, a Bluetooth® connector, an infrared connector, a cellular connector, etc.) configured to communicate over a communication network.

The one or more input interfaces 714 may be, or may include, any number of different types of input units, input circuits, and/or input components that enable the one or more processors 711 to communicate with the one or more input devices 716. Similarly, the one or more output interfaces 715 may be, or may include, any number of different types of input units, input circuits, and/or input components that enable the one or more processors 711 to communicate the one or more output devices 717. In some embodiments, the one or more input interfaces 714 and the one or more output interfaces 715 may be combined into input/output (I/O) units, I/O circuits, and/or I/O components. The one or more input devices 716 may be, or may include, keyboards and/or keypads, interactive screens (e.g., touch screens), navigation devices (e.g., a mouse, a trackball, a capacitive touch pad, a joystick, etc.), microphones, buttons, communication interfaces, etc. The one or more output devices 717 may be, or may include display units (e.g., display screens, receipt printers, etc.), speakers, etc. The one or more input interfaces 714 and/or the one or more output interfaces 716 may also be, or may include one or more digital applications (e.g., local graphical user interfaces (GUIs)).

The one or more digital applications may be, or may include, web-based applications, mobile applications, and/or the like. In some embodiments, the one or more digital applications may be stored on the one or more memories 712. In some embodiments, the one or more digital applications may establish a host-client connection between the application server as the host and a user device (e.g., a desktop, a laptop, a smartphone, a tablet, a wearable, etc.) as the client. In some embodiments, the one or more digital applications may include instantiations of AI-based programs, such as chatbots, to perform one or more aspects of the digital application (e.g., prompts to the user to receive data, handling of data with other machine learning models, processing of data, etc.).

The one or more databases 722 may be, or may include, one or more data repositories and/or the like. For example, the one or more databases 722 may store the training data used to train a machine learning model described herein.

The one or more communication controllers 731 and/or the one or more machine learning controllers 741 may be, or may include, computer-readable, executable instructions that may be stored in the one or more memories and/or performed by the one or more processors 711. The computer-readable, executable instructions of the one or more communication controllers 731 and/or the one or more machine learning controllers 741 may be stored on and/or performed by specifically designated hardware (e.g., micro controllers, microchips, etc.) which may have functionalities similar to the one or more memories and/or the one or more processors 711. The computer-readable, executable instructions of the one or more communication controllers 731 may be configured to send and/or receive electronic data. The computer-readable, executable instructions of the one or more machine learning controllers 741 may be configured to train, validate, and/or develop a machine learning model. The one or more communication controllers 731 and/or the one or more machine learning controllers 741 may work independently and/or in conjunction with one another.

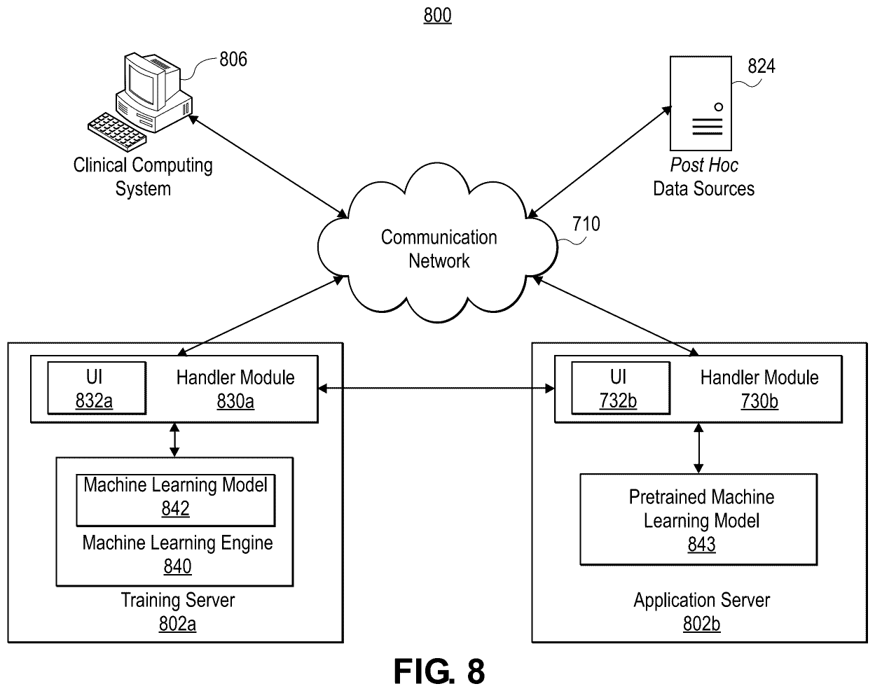

FIG. 8 depicts an exemplary computer environment 800 for the implementation of the methods and systems described herein. The computer environment 800 may include a training server 802a (e.g., the exemplary server 702), an application server 802b (e.g., the exemplary server 702), a clinical computing system 806, a communication network 810, and/or one or more post hoc data sources 824. The training server 802a may include a handler module 830a and/or a machine learning engine 840. The handler 830a module may include a UI 832a. The machine learning engine 840 may develop and/or store a machine learning model 842. The training server 802a may be, or may include, a portion of a memory unit (e.g., the one or more memories 712) configured to store software and/or computer-executable instructions that, when executed by a processing unit (e.g., the one or more processors 711), may train, validate, and/or otherwise develop the machine learning model 842 to (i) predict human health aspects and/or (ii) associate microorganisms to human biological functions.

The application server 802b may include a handler module 830b and/or a pretrained machine learning model 843 (e.g., the machine learning model 842 trained and/or validated by the training server 802a). The handler module 830b may include a UI 832b. The application server 802b may be, or may include, a portion of a memory unit (e.g., the one or more memories 712) configured to store software and/or computer-executable instructions that, when executed by a processing unit (e.g., the one or more processors 711), may cause the one or more of the above-described components to (i) predict human health aspects and/or (ii) associate microorganisms to human biological functions. In some embodiments, the training server 802a and the application server 802b are the same server.

In operation, the training server 802a may train, validate, and/or otherwise develop the machine learning model 842 based upon one or more sets of training data. In some embodiments, the training data is gathered from (i) one or more databases stored on the training server 802a (e.g., the one or more databases 722), (ii) the clinical computing system 806, one or more databases external to the training server 802a (e.g., the one or more post hoc data sources 824), and/or a user device. In some embodiments, the machine learning model 842 may be a binary classification model, such as a CNN, a logistic regression model, a naïve Bayes model, a support vector machine (SVM) model, and/or the like. For example, the binary classifications may be either “pregnancy not resulting in early preterm birth” as a first classification and “pregnancy resulting in early preterm birth” as a second classification. In some embodiments, the machine learning model 842 may be a predictive model. For example, the machine learning model 842 may be able to predict the BMI of an individual based upon their phylotyped gut microorganisms.

Once the training server 802a initially trains and/or initially develops the machine learning model 842, the training server 802a may then validate the machine learning model 842. In some embodiments, the training server 802a segments out a set of validation data that may be from the corpus of training data to use when validating model performance. In these embodiments, the training data may be divided into a ratio of training data and validation data (e.g., 80% training data and 20% validation data). When the machine learning model 842 satisfies a validation metric (e.g., accuracy, recall, area under curve (AUC), etc.) when applied to the validation data, the machine learning model 842 may be implemented as the pretrained machine learning model 843 used by the application server 802b. However, if the machine learning model 842 does not satisfy the validation metrics, the training server 802a may continue training the machine learning model 843 using additional training data.

In operation, in some embodiments, the application server 802b may establish a communicative connection with the user device, the clinical computing system 806 and/or one or more external databases (e.g., the one or more post hoc data sources 824) via a communication network 810. The communication network 810 may be, or may include, the internet, a local area network (LAN), a metropolitan area network (MAN), a wide area network (WAN), a wired network, a Wi-Fi network, a cellular network, a wireless network, a private network, a virtual private network, etc. In some embodiments, establishing the connection may include a user of the user device signing into an account stored with the application server 802b. In some embodiments, establishing the connection may include navigating to a website and/or a web application hosted by the application server 802b. In these embodiments, the user device, as a client, may establish a client-host connection to the application server 802b, as a host. Additionally or alternatively, the user device may establish the client-host connection via an application run on the user device. In some embodiments, the connection may be through either a third party connection (e.g., an email server) or a direct peer-to-peer (P2P) connection/transmission.

The application server 802b may route input data received over the communication network to the handler module 832b. The input data may be a set of phylotyped microbiological data. The handler module 832b may forward the input data to pretrained machine learning model 843, which may output a determination (e.g., a predicted BMI of an individual, a classification if a pregnancy will result in an early preterm birth, etc.). The resulting determination may be returned to the handler module 830b which may in turn present the resulting determination to the user via the user device. In addition to applying the pretrained machine learning model 843, the application server 802b may also perform any of the other methods and systems described herein, including generating phylogenetic trees, phylogenetically placing alleles onto existing phylogenetic trees, performing the divide-and-conquer strategy to generate the phylotypes, generating microbiome atlases, etc.

In some embodiments, the handler module 830a and/or handler module 830b may implement interactive UIs 832a and 832b, respectfully (e.g., a web-based interface, mobile application, etc.) that may be presented by the user device. In particular, the interactive UIs 832a and 832b may be configured to enable the user to submit the training data, the validation data, and/or the input data. In some embodiments, the handler module 832a and 832b may work in conjunction with or be configured to include a chatbot to receive any data from the user.

It should be appreciated that while specific elements, processes, devices, and/or components are described as part of the training server 802a and/or the application server 802b, other elements, processes, devices and/or components are contemplated.

### Exemplary Method

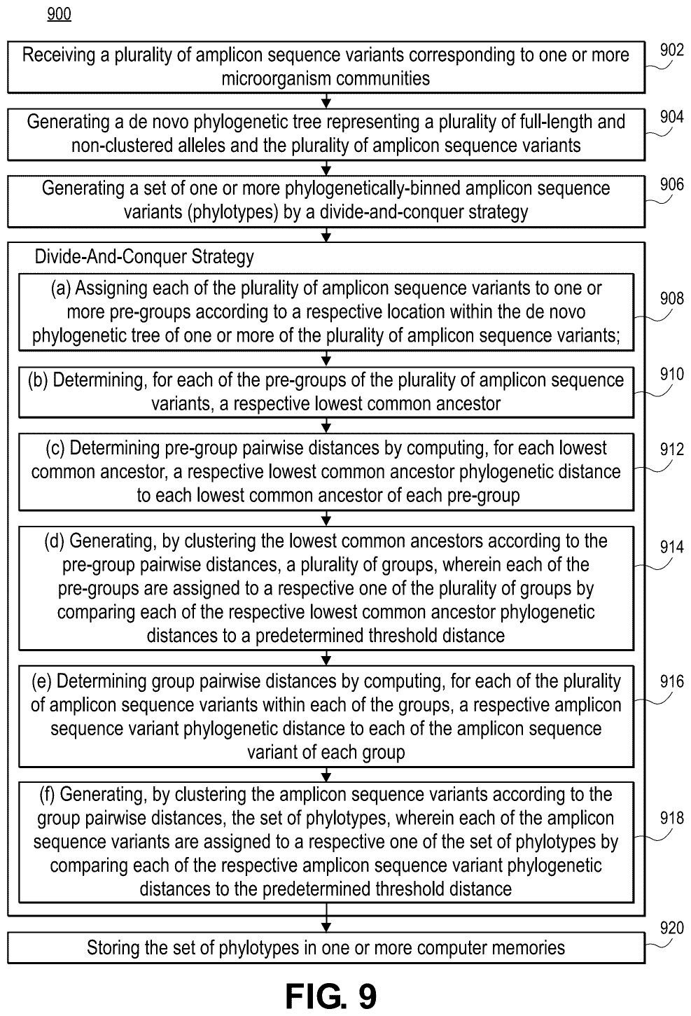

FIG. 9 depicts a block diagram of an exemplary computer-implemented method 900 for a generation of improved taxonomy-independent, generalizable features, according to some aspects. The method 900 may employ any of the techniques, methods, and systems described herein with respect to FIGS. 1A-8.

The method 900 may include (1) receiving, via one or more processors, a plurality of amplicon sequence variants corresponding to one or more microorganism communities (block 902).

The method 900 may include (2) generating, via one or more processors, a de novo phylogenetic tree representing a plurality of full-length and non-clustered alleles and the plurality of amplicon sequence variants (block 904).

The method 900 may include (3) generating, via one or more processors, a set of one or more phylogenetically-binned amplicon sequence variants (phylotypes) by a divide-and-conquer strategy (block 906).

The divide-and-conquer strategy of the method 900 may include (a) assigning each of the plurality of amplicon sequence variants to one or more pre-groups according to a respective location within the de novo phylogenetic tree of one or more of the plurality of amplicon sequence variants (block 908).

The divide-and-conquer strategy of the method 900 may also include (b) determining, for each of the pre-groups of the plurality of amplicon sequence variants, a respective lowest common ancestor (block 910).

The divide-and-conquer strategy of the method 900 may also include (c) determining pre-group pairwise distances by computing, for each lowest common ancestor, a respective lowest common ancestor phylogenetic distance to each lowest common ancestor of each pre-group (block 912).

The divide-and-conquer strategy of the method 900 may also include (d) generating, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance (block 914).

The divide-and-conquer strategy of the method 900 may also include (e) determining group pairwise distances by computing, for each of the plurality of amplicon sequence variants within each of the groups, a respective amplicon sequence variant phylogenetic distance to each of the amplicon sequence variants of each group (block 916).

The divide-and-conquer strategy of the method 900 may also include (f) generating, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance (block 918).

The method 900 may include (4) storing, via one or more processors, the set of phylotypes in one or more computer memories (block 920).

The method 900 may include additional, less, or alternate actions, including those discussed elsewhere herein.

Additionally or alternatively, in some embodiments, the predetermined threshold distance may be 0.1.

Additionally or alternatively, in some embodiments, the one or more microbial communities of the method 900 may be real world gut bacteria and/or the set of previously unseen amplicon sequence variants are derived in silico.

Additionally or alternatively, in some embodiments, the steps of the divide-and-conquer strategy of the method 900 may be performed in parallel via recursion and/or via two or more processors.

Additionally or alternatively, in some embodiments, each of the plurality of amplicon sequence variants of the method 900 may correspond to a respective one of a plurality of individuals.

Additionally or alternatively, in some embodiments, the method 900 may further include receiving, a new plurality of amplicon sequence variants corresponding to the one or more microbial communities, wherein each of the new plurality of amplicon sequence variants corresponds to a respective one of a plurality of previously unseen individuals and/or assigning, by the one or more processors, a phylotype to each of the new plurality of amplicon sequence variants using the divide-and-conquer strategy, wherein newly generated phylotypes are added to the set of phylotypes.

Additionally or alternatively, in some embodiments, the method 900 may further include generating, by the one or more processors, an input vector that may include the new plurality of amplicon sequence variants and the set of phylotypes; applying, by the one or more processors, a developed machine learning model to the input vector to generate one or more of: (i) a prediction of a medical aspect of a previously unseen individual, (ii) an association between the input vector and one or more human biological functions, and/or (iii) an enterotype classification of the one or more microbial communities; and/or storing, via the one or more processors, an output of the developed machine learning model in one or more computer memories.

Additionally or alternatively, in some embodiments, the medical aspect of a patient of the method 900 may be body-mass-index and applying the developed machine learning model to the input vector to generate a prediction of a body-mass-index of a patient may include receiving, by the one or more processors, a training set of body-mass-indices corresponding to a respective one of the plurality of individuals, generating, by the one or more processors, a training input vector may include (i) the plurality of amplicon sequence variants and (ii) the set of phylotypes, excluding, by the one or more processors, a portion of the training input vector and a corresponding portion of the set body-mass-indices based upon the plurality of individuals, training, by the one or more processors using a non-excluded portion of the training input vector and a non-excluded portion of the set of indices, the machine learning model to predict a body-mass-index of an individual, and/or validating, by the one or more processors, the developed machine learning model with an excluded portion of the training input vector and an excluded portion of the set of indices.

### Additional Considerations

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, some embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules may provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may, at a later time, access the memory device to retrieve and process the stored output. Modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

Unless specifically stated otherwise, discussions herein using words such as “receiving,” “analyzing,” “generating,” “creating,” “storing,” “deploying,” “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other.

As used herein any reference to “some embodiments” means that a particular element, feature, structure, or characteristic described in connection with the embodiment may be included in at least one embodiment. The appearances of the phrase “some embodiments” in various places in the specification are not necessarily all referring to the same embodiment. In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112 (f) unless traditional means-plus-function language is expressly recited, such as “means for” or “step for” language being explicitly recited in the claim(s).

This detailed description is to be construed as exemplary only and does not describe every possible embodiment, as describing every possible embodiment would be impractical, if not impossible. One could implement numerous alternate embodiments, using either current technology or technology developed after the filing date of this application. Upon reading this disclosure, those of skill in the art will appreciate still additional alternative structural and functional designs using the disclosed principles herein.

Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein. It is therefore intended that the above-described detailed description be regarded as illustrative rather than limiting, and that it be understood that it is the following claims, including all equivalents, that are intended to define the spirit and scope of this invention.

What is claimed:

1. A computer-implemented method for a generation of improved taxonomy-independent, generalizable features, the method comprising:
receiving, via one or more processors, a plurality of amplicon sequence variants corresponding to one or more microorganism communities;
generating, via one or more processors, a de novo phylogenetic tree representing a plurality of full-length and non-clustered alleles and the plurality of amplicon sequence variants;
generating, via one or more processors, a set of one or more phylogenetically-binned amplicon sequence variants (phylotypes) by a divide-and-conquer strategy including the steps of:
(a) assigning each of the plurality of amplicon sequence variants to one or more pre-groups according to a respective location within the de novo phylogenetic tree of one or more of the plurality of amplicon sequence variants;
(b) determining, for each of the pre-groups of the plurality of amplicon sequence variants, a respective lowest common ancestor;
(c) determining pre-group pairwise distances by computing, for each lowest common ancestor, a respective lowest common ancestor phylogenetic distance to each lowest common ancestor of each pre-group;
(d) generating, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance;
(e) determining group pairwise distances by computing, for each of the plurality of amplicon sequence variants within each of the groups, a respective amplicon sequence variant phylogenetic distance to each of the amplicon sequence variants of each group; and
(f) generating, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and
storing, via one or more processors, the set of phylotypes in one or more computer memories.

2. The computer-implemented method of claim 1, wherein the predetermined threshold distance is 0.1.

3. The computer-implemented method of claim 1, wherein:
each of the plurality of amplicon sequence variants correspond to a respective one of a plurality of individuals, and
the method further comprises:
receiving, a new plurality of amplicon sequence variants corresponding to the one or more microorganism communities, wherein each of the new plurality of amplicon sequence variants corresponds to a respective one of a plurality of previously unseen individuals; and
assigning, by the one or more processors, a phylotype to each of the new plurality of amplicon sequence variants using the divide-and-conquer strategy, wherein newly generated phylotypes are added to the set of phylotypes.

4. The computer-implemented method of claim 3, the method further comprising:
generating, by the one or more processors, an input vector comprising the new plurality of amplicon sequence variants and the set of phylotypes;
applying, by the one or more processors, a developed machine learning model to the input vector to generate one of: (i) a prediction of a medical aspect of a previously unseen individual, (ii) an association between the input vector and one or more human biological functions, or (iii) an enterotype classification of the one or more microorganism communities; and
storing, via the one or more processors, an output of the developed machine learning model in one or more computer memories.

5. The computer-implemented method of claim 4, wherein:
the medical aspect of a patient is body-mass-index, and
applying the developed machine learning model to the input vector to generate a prediction of a body-mass-index of a patient comprises:
receiving, by the one or more processors, a training set of body-mass-indices corresponding to a respective one of the plurality of individuals;
generating, by the one or more processors, a training input vector including (i) the plurality of amplicon sequence variants and (ii) the set of phylotypes;
excluding, by the one or more processors, a portion of the training input vector and a corresponding portion of the set body-mass-indices based upon the plurality of individuals; and
training, by the one or more processors using a non-excluded portion of the training input vector and a non-excluded portion of the set of indices, the machine learning model to predict a body-mass-index of an individual.

6. The computer-implemented method of claim 5, further comprising:
validating, by the one or more processors, the developed machine learning model with an excluded portion of the training input vector and an excluded portion of the set of indices.

7. The computer-implemented method of claim 6, wherein the one or more microorganism communities are real world gut bacteria.

8. The computer-implemented method of claim 6, wherein the set of previously unseen amplicon sequence variants are derived in silico.

9. The computer-implemented method of claim 1, wherein the steps of the divide-and-conquer strategy are performed in parallel via recursion.

10. The computer-implemented method of claim 1, wherein the steps of the divide-and-conquer strategy are performed in parallel via two or more processors.

11. A computer system for a generation of improved taxonomy-independent, generalizable features, the system comprising:
one or more processors;
a non-transitory program memory coupled to the one or more processors and storing executable instructions that, when executed by the one or more processors, cause the computer system to:
receive a plurality of amplicon sequence variants corresponding to one or more microorganism communities;
generate a de novo phylogenetic tree representing a plurality of full-length and non-clustered alleles and the plurality of amplicon sequence variants;
generate a set of one or more phylogenetically-binned amplicon sequence variants (phylotypes) by a divide-and-conquer strategy that cause the one or more processors to:
(a) assign each of the plurality of amplicon sequence variants to one or more pre-groups according to a respective location within the de novo phylogenetic tree of one or more of the plurality of amplicon sequence variants;
(b) determine, for each of the pre-groups of the plurality of amplicon sequence variants, a respective lowest common ancestor;
(c) determine pre-group pairwise distances by computing, for each lowest common ancestor, a respective lowest common ancestor phylogenetic distance to each lowest common ancestor of each pre-group;
(d) generate, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance;
(e) determine group pairwise distances by computing, for each of the plurality of amplicon sequence variants within each of the groups, a respective amplicon sequence variant phylogenetic distance to each of the amplicon sequence variants of each group; and
(f) generate, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and
store the set of phylotypes in one or more computer memories.

12. The computer system of claim 11, wherein the predetermined threshold distance is 0.1.

13. The computer system of claim 11, wherein:
each of the plurality of amplicon sequence variants correspond to a respective one of a plurality of individuals, and
the computer system is further configured to:
receive a new plurality of amplicon sequence variants corresponding to the one or more microorganism communities, wherein each of the new plurality of amplicon sequence variants corresponds to a respective one of a plurality of previously unseen individuals; and
assign a phylotype to each of the new plurality of amplicon sequence variants using the divide-and-conquer strategy, wherein newly generated phylotypes are added to the set of phylotypes.

14. The computer system of claim 13, wherein the computer system is further configured to:
generate an input vector comprising the new plurality of amplicon sequence variants and the set of phylotypes;
apply a developed machine learning model to the input vector to generate one of: (i) a prediction of a medical aspect of a previously unseen individual, (ii) an association between the input vector and one or more human biological functions, or (iii) an enterotype classification of the one or more microorganism communities; and
store an output of the developed machine learning model in one or more computer memories.

15. The computer system of claim 14, wherein:
the medical aspect of a patient is body-mass-index, and
applying the developed machine learning model to the input vector to generate a prediction of a body-mass-index of a patient causes the computer system to:
receive a training set of body-mass-indices corresponding to a respective one of the plurality of individuals;
generate a training input vector including (i) the plurality of amplicon sequence variants and (ii) the set of phylotypes;
exclude a portion of the training input vector and a corresponding portion of the set body-mass-indices based upon the plurality of individuals; and
train, using a non-excluded portion of the training input vector and a non-excluded portion of the set of indices, the machine learning model to predict a body-mass-index of an individual.

16. The computer system of claim 15, wherein the computer system is further configured to:
validate the developed machine learning model with an excluded portion of the training input vector and an excluded portion of the set of indices.

17. The computer system of claim 16, wherein the one or more microorganism communities are real world gut bacteria.

18. The computer system of claim 16, wherein the set of previously unseen amplicon sequence variants are derived in silico.

19. The computer system of claim 11, wherein the steps of the divide-and-conquer strategy are performed in parallel via one or more of (i) recursion or (ii) two or more processors.

20. A tangible, non-transitory computer-readable medium storing executable instructions for a generation of improved taxonomy-independent, generalizable features, the instructions, when executed by one or more processors of a computer system, cause the computer system to:
receive a plurality of amplicon sequence variants corresponding to one or more microorganism communities;
generate a de novo phylogenetic tree representing a plurality of full-length and non-clustered alleles and the plurality of amplicon sequence variants;
generate a set of one or more phylogenetically-binned amplicon sequence variants (phylotypes) by a divide-and-conquer strategy that cause the one or more processors to:
(a) assign each of the plurality of amplicon sequence variants to one or more pre-groups according to a respective location within the de novo phylogenetic tree of one or more of the plurality of amplicon sequence variants;
(b) determine, for each of the pre-groups of the plurality of amplicon sequence variants, a respective lowest common ancestor;
(c) determine pre-group pairwise distances by computing, for each lowest common ancestor, a respective lowest common ancestor phylogenetic distance to each lowest common ancestor of each pre-group;
(d) generate, by clustering the lowest common ancestors according to the pre-group pairwise distances, a plurality of groups, wherein each of the pre-groups are assigned to a respective one of the plurality of groups by comparing each of the respective lowest common ancestor phylogenetic distances to a predetermined threshold distance;
(e) determine group pairwise distances by computing, for each of the plurality of amplicon sequence variants within each of the groups, a respective amplicon sequence variant phylogenetic distance to each of the amplicon sequence variants of each group; and
(f) generate, by clustering the amplicon sequence variants according to the group pairwise distances, the set of phylotypes, wherein each of the amplicon sequence variants are assigned to a respective one of the set of phylotypes by comparing each of the respective amplicon sequence variant phylogenetic distances to the predetermined threshold distance; and
store the set of phylotypes in one or more computer memories.
